(19) United States
(12) Patent Application Publication
(10) Pub. No.: US 2023/0029363 A1
(43) Pub. Date: Jan. 26, 2023

(54) PRODUCTION OF RECOMBINANT PROTEINS USING FAH AS THE SELECTION MARKER

(71) Applicant: Preclinics Gesellschaft für präklinische Forschung mbH, Potsdam (DE)

(72) Inventor: Jonas Füner, Potsdam (DE)

(21) Appl. No.: 17/757,131

(22) PCT Filed: Dec. 14, 2020
(86) PCT No.: PCT/EP2020/086028

§ 371 Date: Jun. 9, 2022

(30) Foreign Application Priority Data
Dec. 12, 2019 (DE) 10 2019 134 122.1

(51) Int. Cl. C12N 5/071; C12N 15/52

(52) U.S. Cl. CPC C12N 5/067; C12N 15/52; C12N 2015/8518

(57) ABSTRACT

The invention relates to a gene construct comprising at least two nucleic acid sequences, wherein one of the nucleic acid sequences encodes FAH and a second nucleic acid sequence encodes a protein to be produced. This makes it possible to use FAH as a selection marker for the production of recombinant proteins, in particular antibodies. The invention further relates to plasmids, vectors or hepatocytes comprising the gene construct. Furthermore, the invention relates to a method for producing recombinant proteins in FAH(−/−) non-human mammals using the gene construct and FAH as a selection marker.

### BACKGROUND TO THE INVENTION

Fumarylacetoacetate hydrolase (FAH) deficiency is a rare disorder called tyrosinemia type1. The deficiency causes the tyrosine metabolites fumaryl acetoacetate and maleyl acetoacetate to fail to be degraded and thus accumulate and are then converted to the toxic substances succinyl acetoacetate and succinyl acetone. Toxic concentrations are quickly reached, particularly in hepatocytes.

Administration of nitisinone (2-(2-nitro-4-trifluoromethyl-benzoyl)-1,3-cyclohexanedione) also known as NTBC blocks the enzyme 4-hydroxyphenylpyruvate dioxigenase, which prevents the formation of toxic substrates.

Tyrosinemia is a disease for which cell or gene therapy could provide a cure. Therefore, transgenic FAH-deficient animal models have been developed to test such treatments. It has been shown in these studies that by introducing healthy liver cells into the livers of FAH knock-out (FAH k.o.) animals, it is possible to cause the FAH-deficient hepatocytes to die and be completely replaced by the healthy cells. Similarly, instead of using normal healthy liver cells, it was possible to use those that had been successfully transferred with the FAH gene. Subsequently, NTBC treatment was discontinued and the FAH-deficient hepatocytes died and were completely replaced by the treated cells.

This has led to the development of a further field of application. This concerns a humanized animal model. If an animal has both FAH deficiency and immunodeficiency, it is possible to implant human hepatocytes into it and thus generate an animal model with human liver metabolism. This is a valuable model for some pharmacological tests.

Since hepatocytes are relatively challenging to maintain in cell culture, this model is also used for primary cell culture of human hepatocytes. In this case, the hepatocytes are then taken from the liver of the FAH k.o. animal treated with human hepatocytes.

Many proteins of importance to humans and animals can only be produced recombinantly in mammalian cells, as only these are able to fold the proteins correctly, or to link their chains, or to enable the corresponding glycosylation. Since the cultivation of mammalian cells is relatively complex compared to yeast and bacterial culture, the production of certain proteins in animals has been considered since the early 1990s. For this purpose, for example, transgenic animals were produced, which then secreted the recombinant protein into, for example, milk. However, the production of large transgenic animals, such as sheep, goats or cattle, is expensive and requires a lot of time and effort. Therefore, this production is only worthwhile for a very large demand, which can be planned in the long term. Strategies involving somatic gene transfer for the production of recombinant proteins in animals have not yet achieved economic viability. So far, the effort is not economically commensurate to the protein yield.

Antibodies have been obtained from animals for over a hundred years. Therefore, there are numerous technologies and protocols for the generation and purification of antibodies from animals. The production of recombinant antibodies in animals is also particularly promising because they only lead to toxic effects in the event of reactivity with a protein in the production host. This can be investigated and ruled out in advance.

It is known from prior art that human hepatocytes can be transplanted into FAH k.o. animals (Ramaswamy et al, “Autologous and herterologous cell therapy for hemophilia B toward functional restoration of factor IX” Cell Rep. 20118, May 01; 23(5): 1565 -1580). These cells can expand as a result of interrupting NTBC administration. The experiments are aimed at investigating possibilities for cell therapy. The organisms are not used to produce recombinant proteins or antibodies.

The prior art also includes EP256082 B, which describes a method for expanding human hepatocytes in vivo. In this method, human hepatocytes are transplanted into FAH-deficient pigs and their expansion is regulated by controlling NTBC administration. The animals are used to select agents for the treatment of human liver disease. However, use as a production host for recombinant proteins is not contemplated.

Patent application PCT/US2003/029251 describes a method of implanting foreign cells into fetal animal organisms and providing them with suitable growth conditions by selectively destroying the body's own cells. However, this method is difficult to carry out, uneconomical and also very stressful for the animals used for production. In the production of recombinant proteins in an animal organism, it is important that, on the one hand, the animal (the production organism) is only minimally stressed and, on the other hand, the effectiveness of the method is so clearly superior to production in cell culture that the use of the animals can be ethically justified.

An interesting method that induces fumarylacetoacetate hydrolase deficiency and uses it to select transfected liver cells was developed by Sean Nygaard, Markus Grompe et al. (Nygaard, Sean et al. “A universal system to select gene-modified hepatocytes in vivo.” Science translational medicine vol. 8,342 (2016): 342ra79. doi:10.1126/scitranslmed.aad8166 and PCT/US2019/029890). The background of this invention is gene therapy application for the treatment of diseases. This method is not suitable for the recombinant production of proteins in animal hosts because the frequent injections of CEHPOBA are, on the one hand, quite expensive and, on the other hand, stressful for the production host.

Therefore, there is a need for further production methods for recombinant proteins and antibodies that achieve high yields, at low cost and low stress to the animals.

### DETAILED DESCRIPTION OF THE INVENTION AND EXAMPLES

The problem underlying the invention is solved by the independent claims. Advantageous embodiments are found in the dependent claims.

In a first preferred embodiment, the invention relates to a gene construct comprising at least two nucleic acid sequences, wherein one of the nucleic acid sequences encodes FAH and a second nucleic acid sequence encodes a protein to be produced.

The special feature of the invention is that the gene constructs for recombinant production of proteins, in addition to the gene of the protein to be produced, also use the FAH gene as a selection marker.

The amino acid sequence for FAH (SEQ ID NO 1) is preferably as follows:

MLGFGRRRLFSALLQVQKRPCQPSRNMRLVQFQAPHLEEPHLGLESGVG
GGVVDLNAFDSTLPKTMVQFLEQGETTLSVARRALATQLPVIPRSQVTF
LAPVTRPDKVICVGLNYADHCQEQNVRVPKSPIIFSKFSSSIVGPYDEI
ILPPESKEVDWEVEMAVVIGKKGKHIKATDVMAHVAGFTVAHDVSARDW
QMRNGKQWLLGKTFDTFCPLGPALVTKDTIADPHNLKICCRVNGEVVQS
SNTNQMVFKTEYLIAWVSQFVTLYPGDLLLTGTPPGVGMFRKPPVFLKK
GDEVQCEIEELGVIINKVV

Preferably, therefore, the gene construct of the invention comprises a nucleic acid sequence encoding SEQ ID NO 1. Also preferably, the gene construct comprises a nucleic acid sequence that encodes an amino acid sequence that is homologous or functionally analogous to SEQ ID NO 1. Preferably there is at least 80% homology, more preferably at least 90% homology, most preferably 95% homology.

The nucleic acid sequence encoding SEQ ID NO1 may also be codon-optimized depending on the production host selected. The person skilled in the art is able to perform this codon optimization independently without having to take an inventive step.

Furthermore, a gene for the recombinant protein to be produced is included in the gene construct. Different promoters can be used. As an example, the FAH nucleic acid sequence may be provided with the ubiquitous CMV promoter and the nucleic acid sequence for the recombinant protein may be provided with an albumin promoter. The person skilled in the art is able to select a suitable promoter without having to take an inventive step. In accordance with the invention, it is also possible to provide the FAH gene and the gene of the protein to be produced with one and the same promoter and to share the gene of the protein to be produced with a 2A peptide to the FAH gene.

The protein to be produced, encoded by the second nucleic acid sequence, is preferably a human protein, in particular a human antibody. This means that these proteins or antibodies either occur in humans or can be used in humans. However, non-human proteins and antibodies may also be of particular interest for certain applications, so that the invention is not limited thereto.

A “protein to be produced” is a protein specifically selected or desired by the user or experimenter. A protein to be produced may be a naturally occurring protein or an artificial protein, such as a fusion protein. With respect to the invention, non-limiting examples of such proteins to be produced may include: Antibodies, preferably human antibodies, for passive immunization, such as IgG which binds to the RBD of the SARS-CoV-2 spike protein and neutralizes the virus, or IgG which binds to fragment C of the tetanus toxin and thus prevents cell entry of the toxin. Also, the protein to be produced may be antibodies for clinical diagnostics, which need to be obtained on a large scale, such as rabbit antibodies against human immunoglobulins (IgG, IgA, IgM, IgE), or goat antibodies against alphal -antitrypsin and other antibodies from various species against, for example, transferrin, ferritin, C-reactive protein, albumin, various complement factors (C3, C4 Cl inhibitor, etc.). A protein to be produced may also be one of therapeutic, vaccine, agricultural or veterinary interest. Proteins of therapeutic interest may include, in particular, enzymes, blood derivatives, hormones, lymphokines (interleukins, interferons, TNF, etc.), growth factors, neurotransmitters or their precursors or synthesis enzymes, trophic factors (BDNF, CNTF, NGF, IGF, GMF, aFGF, bFGF, NT3, NT5, etc.), apolipoproteins (ApoAI, ApoAIV, ApoE, etc.), dystrophin or a minidystrophin, tumor suppressor genes (p53, Rb, Rapt A, DCC, k-rev, etc.), coagulation factors VII, VIII, IX, etc., or even the whole or a part of a natural or artificial immunoglobulin (Fab, ScFv, etc.). The protein to be produced may also be an antigen or immunogen capable of eliciting an immune response in humans or animals, for the production of vaccines. In particular, it may be antigen peptides specific for bacteria, viruses or tumors. It must be ensured that the therapeutic protein to be produced has no, or at least no harmful effect in the production host. The prerequisite for this is the lowest possible homology of the protein to be produced with the host's own proteins.

This gene construct is preferably used by being introduced into hepatocytes from FAH-deficient animals (production host) via gene transfer, e.g. transfection or transduction. This then results in the cells in the liver that have received the gene construct producing the recombinant protein and secreting it into the blood when a secretory signal is used. In addition, the cells produce FAH, which can then be used as a selection marker. Cells that produce no FAH or no functional FAH accumulate the toxic substances succinyl acetoacetate and succinyl acetone and die. This can only be prevented via balancing substances (e.g. NTBC) that ensure that succinyl acetoacetate and succinyl acetone do not accumulate. When these substances are no longer administered, only cells that have received the FAH gene via gene transfer can still survive. These cells have also simultaneously received the nucleic acid sequence for the protein to be produced. Thus, FAH can be used to select which cells have undergone gene transfer.

Gene transfer as used herein refers to the transfer or transmission of one or more genes or genetic material within the gene construct to a eukaryotic cell. In selective gene transfer, the one or more genes are passed only to a specific cell population (target cell population) or to a tissue, herein preferably liver tissue. Selective gene transfer can be facilitated by the introduction of a targeting domain, e.g. on the viral surface.

It is preferred that the nucleic acid sequence encoding the protein to be produced additionally comprises a signal sequence for secretion. Depending on the intended use, a secretory signal can be used to secrete the recombinant protein into the blood. The selection of the secretory signal depends on the recombinant protein selected.

The following is a non-exhaustive list of possible signal sequences which are particularly suitable for this purpose. These are the amino acid sequences, and the gene construct preferably comprises the respective nucleic acid sequences that encode these amino acid sequences:

| Sequence | | |
|---|---|---|
| origin | ID No | Sequence |
| Mouse | SEQ ID NO 2 | METDTLLLWVLLLWVPGSTGD |
| Ig Kappa | | |
| Human | SEQ ID NO 3 | MGWSCIILFLVATATGVHS |
| IgG2 H | | |
| Human | SEQ ID NO 4 | MDMRVPAQLLGLLLLWLRGARC |
| IgKVIII | | |
| Human | SEQ ID NO 5 | MYRMQLLSCIALSLALVTNS |
| IL-2 | | |
| Gaussia | SEQ ID NO 6 | MGVKVLFALICIAVAEA |
| luc | | |
| Albumin | SEQ ID NO 7 | MKWVTFISLLFSSAYS |
| (HSA) | | |

The person skilled in the art is able to select an appropriate signal sequence for the respective use without having to take an inventive step.

Also suitable are sequences that are homologous or functionally analogous to the sequences mentioned. Preferably there is at least 80% homology, particularly preferably at least 90% homology, most preferably 95% homology. Accordingly, it is preferred that the gene construct comprises a nucleic acid sequence encoding such a homologous signal sequence.

The gene encoding the protein to be produced, with a secretory signal, and the FAH-encoding gene are preferably cloned or inserted into a common vector. This vector is then used to transfect and/or transduce liver cells from the FAH deficient animal.

In a further preferred embodiment, the invention relates to a vector comprising a gene construct according to the invention.

It is preferred that the vector is a viral vector, especially preferably a lentiviral vector.

Thus, the terms “vector” and “viral vector” as used herein preferably refer to viral particles.

In another preferred embodiment, the invention relates to a plasmid comprising a gene construct according to the invention.

In another preferred embodiment, the invention relates to a liver cell into which the gene construct according to the invention has been introduced. Preferably, the liver cell was FAH deficient prior to gene transfer, such that it either lacked the FAH gene or the FAH gene was mutated and ultimately no FAH could be produced.

In another preferred embodiment, the invention relates to a transgenic non-human mammal which has received the gene construct according to the invention by gene transfer and which was an FAH (−/−) animal prior to gene transfer.

In principle, all non-human mammals are conceivable as production hosts. Preferably, the animal is a pig. When pigs are used as production hosts, an advantage arises from the affinity of porcine IgG for protein G and a lack of affinity for protein A, whereas most immunoglobulin G groups from other species have an affinity for protein A or for both protein A and G. Thus, when using pigs as production hosts, the recombinant antibodies can be purified separately from the porcine IgG by protein A binding.

Alternatively, double transgenic production hosts that do not produce immunoglobulins can be used.

Furthermore, it is preferred that the animal is a sheep. Sheep are particularly well suited for the production of serum, as plasmapheresis is easier and thus less stressful for the animals due to the easily accessible vessels.

However, it is also possible to use other non-human mammals, e.g. mouse, rat, rabbit, hare, goat, cow, horse, camel or others. The use of larger animals has the advantage that the amount of antibodies to be produced is greater.

It is preferred that the animal has an immunodeficiency. This has the advantage that an immune reaction against the protein to be produced is less likely.

In another preferred embodiment, the invention relates to a process for the production of recombinant proteins, preferably antibodies comprising the following steps:
a) Providing a non-human FAH (−/−) mammalian production host maintained under NTBC administration,
b) Providing a gene construct, plasmid, vector and/or liver cell according to the invention,
c) Gene transfer, wherein the nucleic acid of the gene construct, plasmid, vector and/or liver cell from step b is introduced into hepatocytes of the production host,
d) Reduction or discontinuation of NTBC administration to expand FAH positive liver cells,
e) Isolation of the recombinant proteins.

Instead of NTBC, another substance can be used that interferes with tyrosine degradation. Possible substances include shRNAs, such as CEHPOBA, or other substances that prevent the accumulation of succinyl acetoacetate and succinyl acetone. Whenever NTBC is mentioned in connection with the invention, such an alternative substance may also be used.

The gene transfer is preferably a transfection or transduction.

Preferably, the gene transfer can be carried out by in vivo transfection of the hepatocytes. For this purpose, there are various methods known to the person skilled in the art which are suitable for the invention. Firstly, hydrodynamic transfection can be used, in which blood is displaced in the liver using the infusion technique and the infusion solution with the vectors perfuses through the liver under increased pressure. It is also possible to use specific lipid nanoparticles in which the vectors are packaged and which are thus taken up directly by the hepatocytes.

However, quite nonspecific transfection can also be performed with systemically or intravenously administered viral vectors, preferably lentiviruses, since there is no disadvantage to transfecting other cell types as well. Due to selection in hepatocytes, the majority of transgene expression will ultimately occur in the liver with any procedure. However, the vectors can also be introduced into the liver by direct injection into the liver tissue. This can also be done percutaneously. When injected directly into liver tissue, transfection efficiency can be increased by in vivo electroporation.

It is preferred that the gene transfer of the liver is performed in newborn production hosts. This has the advantage, especially in larger animals, that the use of NTBCs can be reduced and thus a considerable cost saving is achieved. In all species, however, it is advantageous in that it makes it more likely that the production host will develop immunological tolerance to the recombinant protein being produced.

It is also possible to perform gene transfer prenatally in utero.

It is preferred that discontinuation of NTBC not occur immediately after gene transfer. When to discontinue or reduce NTBC administration depends on both the animal and the gene transfer method. For example, discontinuation can occur one day after gene transfer or several days later. Sufficient time must have elapsed for the nucleic acids of the gene construct to integrate into the genome of the liver cells so that FAH can actually be used as a selection marker.

When NTBC is discontinued after gene transfer, the selection process begins. NBTC blocks the enzyme hydroxyphenylpyruvate dioxygenase upstream of FAH, preventing accumulation of hepatotoxic metabolites. The non-transfected cells die upon withdrawal of NTBC, while all transfected cells, due to co-expression of FAH survive and colonize the liver tissue. As a result, at the end of the selection process, the liver possesses exclusively hepatocytes, which also produce the recombinant protein. Depending on the animal species, this process takes different amounts of time, but is usually completed after a few weeks.

The method uses animals in which the gene for fumarylacetoacetate hydrolase (FAH) is missing or defective.

It is particularly preferred that the proteins are taken from the blood. Thus, proteins, in particular antibodies, can be produced and then isolated in a very simple and stress-free manner for the animal. An advantage of producing recombinant proteins, in particular recombinant antibodies in this system is the extensive experience with obtaining proteins and antibodies from the blood. Here, plasmapheresis can be used to obtain as much and as long as possible recombinant proteins from each production host. Furthermore, the purification protocols and equipment for immunoglobulin precipitation can be used. Thus, purification and further processing on an industrial scale are readily possible without being a major cost issue.

The recombinant proteins are preferably obtained by plasmapheresis. This method is particularly suitable for antibodies, as these are plasma proteins and are thus present in the plasma in their natural matrix.

It is also possible to purify the proteins alternatively or in parallel from the liver, or to obtain and culture hepatocytes that produce the recombinant protein.

It is further preferred that the non-human FAH (−/−) mammal is immunodeficient. Alternatively, immunosuppressive treatment can be performed, e.g., with cyclosporine. However, this is preferable only in certain cases, as this method leads to significantly higher husbandry costs and also to a higher level of stress for the animal.

The invention enables proteins, in particular antibodies, to be produced in large quantities at significantly lower cost and, above all, more quickly than has previously been possible. Such quantities are not possible in cell culture, so the invention is particularly suitable for the production of therapeutic antibodies, e.g. in the event of a pandemic—i.e. when particularly large quantities are required quickly.

In another preferred embodiment, the invention relates to the use of FAH (−/−) non-human mammals for the production of recombinant proteins, in particular antibodies.

It is preferred here that the FAH (−/−) non-human mammals are also immunodeficient.

In this regard, it is further preferred that a gene construct according to the invention be introduced into the FAH (−/−) non-human mammal.

The use of the animals according to the invention, in combination with the gene construct, is very well suited for the rapid and economical production of medium to very large quantities of recombinant proteins, in particular antibodies.

In a preferred embodiment, the invention further relates to a kit comprising a gene construct according to the invention and/or a vector according to the invention and/or a plasmid according to the invention and adjuvants for gene transfer, preferably for transfection and/or transduction.

The teaching according to the application is characterized in particular by the following features:
Departure from that which is technically usual
New problem definition
Existence of a long unsolved urgent need for the solution of the problem solved by the invention.
Development of scientific technology went in a different direction.

In particular, the advantageous embodiments of the invention have at least one or more of the above advantages.

All preferred embodiments described for a particular claim category apply to the other categories as well.

### EXAMPLE

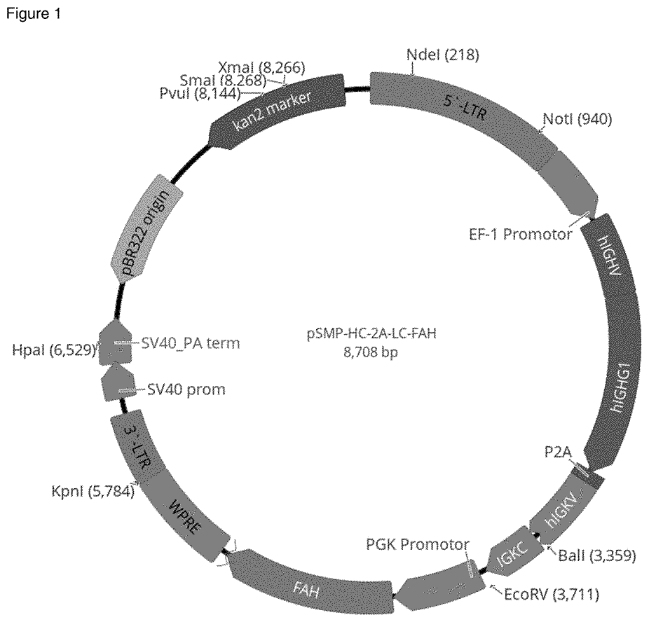

For the planned production of a therapeutic antibody directed e.g. against nucleoprotein of influenza virus A, a lentiviral vector is first produced. The plasmid pSMP-Anti-NP-FAH (FIG. 1) is a lentiviral expression vector with two promoters EF-1α and PGK. The human NP-specific antibody (nucleoprotein of influenza virus A) heavy and light chain sequences are separated with the sequence for a P2A peptide and cloned under the control of an EF-1α promoter. The FAH sequence is cloned downstream of the PGK promoter.

A third-generation lentiviral packaging system (with VSV-G) is set up together with the expression plasmid pSMP anti-NP-FAH for the production of anti-NP-FAH lentiviral vectors.

By transducing hepatocytes from an FAH(−/−) animal with the anti-NP-FAH lentiviral vectors, the expression cassettes of anti-NP and FAH are integrated into the genome of the transduced cells. In an FAH(−/−) animal model, hepatocytes are selected by the expression of FAH. The anti-NP antibody can now be produced by the selected hepatocytes.

For this purpose, 10⁸ TU of the lentiviral vector is transcutaneously injected into the large liver lobe of 3 to 4 week old FAH(−/−) mice under anesthesia (alternatively, the lentiviral vectors can also be injected intravenously). NTBC administration (4mg/m1 via drinking water) is discontinued one day after transfection, thus initiating the selection process.

After 6 weeks, the antibody can be isolated from serum or liver tissue.

The plasmid contains the following sequences:

Anti-NP sequence

IGHV DNA sequence
SEQ ID NO 8
ATGGAGTTTGGGCTGAGCTGCGTTTTCCTTGTTGCCATTTTTAAAGGTATCGATGTACATTCCGA
GGTGCAGCTGGTGGAGTCTGGGGCTGAGGTGAGGAAGCCTGGGGCCTCAGTGAAGGTCTCCT
GCAAGGCTTCTGGATACACCTTCACCGGCTACTATATTCACTGGGTGCGACAGGCCCCTGGAC
AAGGACTTGAGTGGTTGGGACGGATCAACCCAAACAGTGGCCCATACATTGATGGCACAAAGT
ACGCAGAGAAGTTTCAGGGCCGGGTCACCATGACTAGCGACAGGTCCATTAACACAGCCTACA
TGGAACTGAGCAGGCTGACATCTGACGACACGGCCGTCTATTACTGTGCGAGGGAAGATGTGA
TAGACTCCTACTTTGACTTGTGGAGCCAGGGAACCCTGGTCACCGTCTCCTCAGGC
SEQ ID NO 8 encodes the following amino acid sequence:
SEQ ID NO 9
MEFGLSCVFLVAIFKGIDVHSEVQLVESGAEVRKPGASVKVSCKASGYTFTGYYIHWVRQAPGQGL
EWLGRINPNSGPYIDGTKYAEKFQGRVTMTSDRSINTAYMELSRLTSDDTAVYYCAREDVIDSYFDL
WSQGTLVTVSSG
IGKV DNA sequence:
SEQ ID NO 10
ATGGAAACCCCAGCGCAGCTTCTCTTCCTCCTGCTACTCTGGCTCCCAGTTTCAGATCGTACGG
TACATGGGGATATTGTGATGACCCAGTCTCCACTCTCCCTGCCCGTCACCCCTGGAGAGCCGG
CCTCCATCTCCTGCAGGTCTAGTCAGAGCCTCCTGCATAGTAATGGATACAACTATTTAGATTG
GTACCTGCAGAAGCCAGGGCAGTCTCCACAGCTCCTCATGTATTTGGGTTCTACTCGGGCCTC
CGGGGTCCCTGACAGGTTCAGTGGCAGTGGATCAGGCACAGATTTTACACTGAAAATCAGCAC
AGTGGAGGCTGAGGATGTTGGAATTTATTACTGCATGCAAGCTCTACAAGCTCCGTACACTTTT
GGCCAAGGGACCAAAGTGGATATCAAA
SEQ ID NO 10 encodes the following amino acid sequence:
SEQ ID NO 11:
METPAQLLFLLLLWLPVSDRTVHGDIVMTQSPLSLPVTPGEPASISCRSSQSLLHSNGYNYLDWYLQ
KPGQSPQLLMYLGSTRASGVPDRFSGSGSGTDFTLKISTVEAEDVGIYYCMQALQAPYTFGQGTKV
DIK

Also included is a gene segment homologous to SEQ ID NO 1 or a sequence functionally analogous thereto, preferably 80% homology, more preferably 90% homology, most preferably 95% homology.

FIG. 1 shows the expression plasmid pSMP-Anti-NP-FAH. It is a lentiviral expression vector with two promoters EF-1α and PGK. The human NP-specific antibody (nucleoprotein of influenza virus A) heavy and light chain sequences are separated with the sequence for a P2A peptide and cloned under the control of an EF-1α promoter. The FAH sequence is cloned downstream of the PGK promoter.

1. A gene construct comprising at least two nucleic acid sequences, wherein one of the nucleic acid sequences encodes FAH and a second nucleic acid sequence encodes a protein to be produced.

2. The gene construct according to claim 1, wherein the nucleic acid sequence encoding the protein to be produced additionally comprises a signal sequence for secretion.

3. The gene construct according to claim 1, wherein the protein to be produced is an antibody.

4. A vector comprising a gene construct according to claim 1.

5. A vector according to claim 4, wherein the vector is a viral vector.

6. A plasmid comprising a gene construct according to claim 1.

7. A liver cell into which the gene construct according to claim 1 has been introduced.

8. A transgenic non-human mammal which has received, by gene transfer, the gene construct according to claim 1 and which was an FAH (−/−) animal prior to gene transfer.

9. Method for the production of recombinant proteins comprising the following steps:
(a) obtaining a non-human FAH (−/−) mammalian production host maintained under nitisinone (2-(2-nitro-4-trifluoromethly-benzoyl)-1,3-cyclohexanedione) (NTBC) administration,
(b) obtaining a gene construct according to claim 1,
(c) introducing the gene construct, from step b into hepatocytes of the production host,
(d) reducing or discontinuing NTBC administration to the non-human FAH (−/−) mammalian production host to expand FAH positive liver cells,
(e) isolating the recombinant proteins from the non-human FAH (−/−) mammalian production host.

10. The method according to the claim 9, wherein the recombinant proteins are taken from blood.

11. The method according to claim 9, wherein the non-human FAH (−/−) mammal has an immunodeficiency.

12.-15. (canceled)

16. The method of claim 9, wherein the recombinant proteins are antibodies.

17. The method of claim 9, wherein the recombinant proteins are taken from liver tissue.

18. The method of claim 9, wherein the gene construct is contained within a plasmid or a vector.

19. The method of claim 9, wherein the gene construct is contained within a liver cell and wherein the liver cell is administered to the non-human FAH (−/−) mammalian production host.

20. The method according to claim 18, wherein the recombinant proteins are taken from blood or liver tissue.

21. The method according to claim 18, wherein the non-human FAH (−/−) mammal has an immunodeficiency.

22. The method according to claim 19, wherein the recombinant proteins are taken from blood or liver tissue.

23. The method according to claim 19, wherein the non-human FAH (−/−) mammal has an immunodeficiency.


SEQUENCE LISTING (11 sequences)

SEQ ID NO: 1 - protein, 313 aa, Artificial Sequence
  Polypeptide
MLGFGRRRLFSALLQVQKRPCQPSRNMRLVQFQAPHLEEPHLGLESGVGGGVVDLNAFDSTLPKTMVQFLEQGETTLSVARRALATQLPVIPRSQVTFLAPVTRPDKVICVGLNYADHCQEQNVRVPKSPIIFSKFSSSIVGPYDEIILPPESKEVDWEVEMAVVIGKKGKHIKATDVMAHVAGFTVAHDVSARDWQMRNGKQWLLGKTFDTFCPLGPALVTKDTIADPHNLKICCRVNGEVVQSSNTNQMVFKTEYLIAWVSQFVTLYPGDLLLTGTPPGVGMFRKPPVFLKKGDEVQCEIEELGVIINKVV

SEQ ID NO: 2 - protein, 21 aa, Mouse
METDTLLLWVLLLWVPGSTGD

SEQ ID NO: 3 - protein, 19 aa, Artificial Sequence
  Peptide
MGWSCIILFLVATATGVHS

SEQ ID NO: 4 - protein, 22 aa, Artificial Sequence
  Peptide
MDMRVPAQLLGLLLLWLRGARC

SEQ ID NO: 5 - protein, 20 aa, Artificial Sequence
  Peptide
MYRMQLLSCIALSLALVTNS

SEQ ID NO: 6 - protein, 17 aa, Artificial Sequence
  Peptide
MGVKVLFALICIAVAEA

SEQ ID NO: 7 - protein, 16 aa, Artificial Sequence
  Peptide
MKWVTFISLLFSSAYS

SEQ ID NO: 8 - DNA, 435 bp, Artificial Sequence
  Anti-NP
atggagtttgggctgagctgcgttttccttgttgccatttttaaaggtatcgatgtacattccgaggtgcagctggtggagtctggggctgaggtgaggaagcctggggcctcagtgaaggtctcctgcaaggcttctggatacaccttcaccggctactatattcactgggtgcgacaggcccctggacaaggacttgagtggttgggacggatcaacccaaacagtggcccatacattgatggcacaaagtacgcagagaagtttcagggccgggtcaccatgactagcgacaggtccattaacacagcctacatggaactgagcaggctgacatctgacgacacggccgtctattactgtgcgagggaagatgtgatagactcctactttgacttgtggagccagggaaccctggtcaccgtctcctcaggc

SEQ ID NO: 9 - protein, 145 aa, Artificial Sequence
  Anti-NP
MEFGLSCVFLVAIFKGIDVHSEVQLVESGAEVRKPGASVKVSCKASGYTFTGYYIHWVRQAPGQGLEWLGRINPNSGPYIDGTKYAEKFQGRVTMTSDRSINTAYMELSRLTSDDTAVYYCAREDVIDSYFDLWSQGTLVTVSSG

SEQ ID NO: 10 - DNA, 408 bp, Artificial Sequence
  IGKV
atggaaaccccagcgcagcttctcttcctcctgctactctggctcccagtttcagatcgtacggtacatggggatattgtgatgacccagtctccactctccctgcccgtcacccctggagagccggcctccatctcctgcaggtctagtcagagcctcctgcatagtaatggatacaactatttagattggtacctgcagaagccagggcagtctccacagctcctcatgtatttgggttctactcgggcctccggggtccctgacaggttcagtggcagtggatcaggcacagattttacactgaaaatcagcacagtggaggctgaggatgttggaatttattactgcatgcaagctctacaagctccgtacacttttggccaagggaccaaagtggatatcaaa

SEQ ID NO: 11 - protein, 136 aa, Artificial Sequence
  IGKV
METPAQLLFLLLLWLPVSDRTVHGDIVMTQSPLSLPVTPGEPASISCRSSQSLLHSNGYNYLDWYLQKPGQSPQLLMYLGSTRASGVPDRFSGSGSGTDFTLKISTVEAEDVGIYYCMQALQAPYTFGQGTKVDIK